(19) United States
(12) Patent Application Publication
(10) Pub. No.: US 2025/0322930 A1
(43) Pub. Date: Oct. 16, 2025

(54) SYSTEMS AND METHODS FOR PATIENT GUIDED NEUROMODULATION THERAPY

(71) Applicant: Boston Scientific Neuromodulation Corporation, Valencia, CA (US)

(72) Inventors: Lisa Denise Moore, Glendale, CA (US); Mahsa Malekmohammadi, Sherman Oaks, CA (US); Bradley Lawrence Hershey, Carrollton, TX (US)

(21) Appl. No.: 19/171,543

(22) Filed: Apr. 7, 2025

Related U.S. Application Data
(60) Provisional application No. 63/632,932, filed on Apr. 11, 2024.

(51) Int. Cl. G16H 20/40; G16H 10/20; G16H 10/60; G16H 20/10

(52) U.S. Cl. CPC G16H 20/40; G16H 10/20; G16H 10/60; G16H 20/10

(57) ABSTRACT

Systems and methods for patient guided neuromodulation therapy are disclosed. An exemplary system comprises a processing system and a memory. The memory may store instructions executable by the processing system to receive a medication schedule from a patient associated with neuromodulation therapy. The instructions may be executable by the processing system to send queries related to symptoms and side effects experienced by the patient. The instructions may be executable by the processing system to determine a parameter for a period of time of medication efficacy. The instructions may be executable by the processing system to determine a change in the parameter for the period of time.

### CLAIM OF PRIORITY

This application claims the benefit of U.S. Provisional Application No. 63/632,932, filed on Apr. 11, 2024, which is hereby incorporated by reference in its entirety.

### TECHNICAL FIELD

This document relates generally to medical systems, and more particularly, but not by way of limitation, to systems, devices, and methods for patient guided neuromodulation therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc.

A therapy device may be configured to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. In another nonlimiting example, a device, such as a myocardial stimulator and/or neurostimulator, may be configured to treat cardiovascular condition. Settings of the therapy device may be programmed based on observed clinical effects so that the therapy provides desirable intended effects (e.g., reduced tremor, bradykinesia, and dyskinesia for a PD therapy, desirable pain relief or paresthesia coverage for a pain therapy, desirable blood pressure and/rhythms for a cardiovascular therapy) while avoiding undesirable side effects.

### SUMMARY

An example (e.g., “Example 1”) of a system may include a processing system and a memory. The memory may be coupled to the processing system, the memory storing instructions that, when executed by the processing system, cause the processing system to: receive a medication schedule from a patient associated with neuromodulation therapy; send queries related to symptoms and side effects experienced by the patient; determine a parameter for a period of time of medication efficacy; and determine a change in the parameter for the period of time.

In Example 2, the subject matter of Example 1 optionally includes wherein the processing system is configured for use to: determine that the change in the parameter exceeds a threshold change; and in response to the parameter exceeding the threshold change, recommend an adjustment to the neuromodulation therapy.

In Example 3, the subject matter of any one or more of Examples 1-2 optionally include wherein the processing system is configured for use to send at least one query associated with determining a percentage of time while the patient was awake during a period of time that the patient experienced a change in side effects or symptoms.

In Example 4, the subject matter of any one or more of Examples 1-3 optionally include wherein the processing system is configured for use to send the queries related to the symptoms and the side effects at specified time intervals.

In Example 5, the subject matter of any one or more of Examples 1-4 optionally include wherein the processing system is configured for use to: receive responses to the queries; and analyze the responses to determine the change in the parameter.

In Example 6, the subject matter of any one or more of Examples 1-5 optionally include wherein the processing system is configured for use to identify patterns in the parameter of the medication efficacy during different periods of time.

In Example 7, the subject matter of any one or more of Examples 1-6 optionally include wherein the parameter is an average medication efficacy over the period of time.

In Example 8, the subject matter of any one or more of Examples 1-7 optionally include wherein the processing system is configured for use to communicate with a caregiver of the patient to receive caregiver feedback regarding the medication efficacy of the patient.

In Example 9, the subject matter of any one or more of Examples 1-8 optionally include wherein the processing system is configured for use to: determine a threshold change for the parameter of the medication efficacy; and send additional queries to the patient when the parameter exceeds the threshold change in the parameter.

In Example 10, the subject matter of any one or more of Examples 1-9 optionally include wherein the processing system is configured for use to provide reports that comprises at least one of an average on-time per dose, a daily average on-time, a weekly average on-time, a monthly average on-time, and a variance of at least one of the previously mentioned averages.

In Example 11, the subject matter of any one or more of Examples 1-10 optionally include wherein the processing system is configured for use to adjust a frequency of the queries to the patient based on a proximity of a scheduled physician visit or upon request by a user associated with the patient.

In Example 12, the subject matter of any one or more of Examples 1-11 optionally include wherein the processing system is configured for use to receive a sleep schedule from the patient and to use the sleep schedule to determine the parameter for the period of time.

In Example 13, the subject matter of any one or more of Examples 1-12 optionally include wherein: the parameter is a percentage of time; and the processing system is configured for use to: determine that the patient experienced at least one side effect or at least one symptom during the percentage of time during the period of time; and send a query to determine the percentage of time at least one hour before the patient is to go to sleep.

In Example 14, the subject matter of any one or more of Examples 1-13 optionally include wherein the processing system is configured for use to send the queries related to the symptoms and the side effects during a one week period prior to or a one week period subsequent to the patient seeing a physician.

In Example 15, the subject matter of any one or more of Examples 1-14 optionally include wherein the processing system is configured for use to program at least one medical device with a selected stimulation parameter to deliver the stimulation therapy.

Example 16 includes subject matter (such as a method, means for performing acts, machine readable medium including instructions that when performed by a machine cause the machine to perform acts, or an apparatus to perform). The subject matter may include receiving a medication schedule from a patient associated with neuromodulation therapy; sending queries related to symptoms and side effects experienced by the patient; determining a parameter for a period of time of medication efficacy; and determining a change in the parameter for the period of time.

In Example 17, the subject matter of Example 16 optionally includes determining that the change in the parameter exceeds a threshold change; and in response to the parameter exceeding the threshold change, recommending an adjustment to the neuromodulation therapy.

In Example 18, the subject matter of any one or more of Examples 16-17 optionally include sending at least one query associated with determining a percentage of time while the patient was awake during a period of time that the patient experienced a change in side effects or symptoms.

In Example 19, the subject matter of any one or more of Examples 16-18 optionally include sending the queries related to the symptoms and the side effects at specified time intervals.

In Example 20, the subject matter of any one or more of Examples 16-19 optionally include receiving responses to the queries; and analyzing the responses to determine the change in the parameter.

In Example 21, the subject matter of any one or more of Examples 16-20 optionally include identifying patterns in the parameter of the medication efficacy during different periods of time.

In Example 22, the subject matter of any one or more of Examples 16-21 optionally include wherein the parameter is an average medication efficacy over the period of time.

In Example 23, the subject matter of any one or more of Examples 16-22 optionally include receiving a sleep schedule from the patient.

In Example 24, the subject matter of any one or more of Examples 16-23 optionally include hour before the patient goes to bed.

In Example 25, the subject matter of any one or more of Examples 16-24 optionally include sending the queries every other week.

In Example 26, the subject matter of any one or more of Examples 16-25 optionally include sending the queries once a month for patients who are not administered medication.

In Example 27, the subject matter of any one or more of Examples 16-26 optionally include receiving a response from the patient in response to the queries.

In Example 28, the subject matter of Example 27 optionally includes wherein the received response is used to determine one of: an average on-time for each dose administered to the patient, an average on-time percentage per waking hour, or a daily average on-time.

In Example 29, the subject matter of any one or more of Examples 27-28 optionally include wherein the received response is used to determine one of: a weekly average on-time, a monthly average on-time, or a variance of each recording from a determined average.

In Example 30, the subject matter of any one or more of Examples 16-29 optionally include providing a recommendation based on a response to the queries that indicates the patient is trending in a positive direction or a negative direction or remaining stable over time.

Example 31 includes subject matter (such as a method, means for performing acts, machine readable medium including instructions that when performed by a machine cause the machine to perform acts, or an apparatus to perform). The subject matter may include receiving a medication schedule from a patient associated with neuromodulation therapy; sending queries related to symptoms and side effects experienced by the patient; determining a parameter for a period of time of medication efficacy; and determining a change in the parameter for the period of time; and compare the change in the parameter to an average parameter for the patient.

In Example 32, the subject matter of Example 31 optionally includes wherein the instructions are executable by the processor to set a trigger in response to the comparison indicating the change in the parameter is outside a threshold difference from the average parameter.

In Example 33, the subject matter of Example 32 optionally includes wherein the instructions are executable by the processor to provide a recommendation to adjust the neuromodulation therapy based on the set trigger.

In Example 34, the subject matter of any one or more of Examples 32-33 optionally include wherein the instructions are executable by the processor to inform connected users that the trigger was set.

In Example 35, the subject matter of Example 34 optionally includes wherein the instructions are executable by the processor to provide a recommendation to the connected users in response to the trigger being set.

Example 36 includes subject matter (such as a method, means for performing acts, machine readable medium including instructions that when performed by a machine cause the machine to perform acts, or an apparatus to perform). The subject matter may include receiving a trigger signal initiated by the patient; providing the stimulation therapy to the patient using an adjusted stimulation parameter set, the stimulation parameter set is generated by adjusting a stimulation parameter set based on receiving the trigger signal for a specified period of time; and returning to providing the stimulation therapy using the stimulation parameter set after the specified period of time.

In Example 37, the subject matter of Example 36 optionally includes wherein the trigger signal is initiated by the patient in response to an increase in symptoms or side effects.

In Example 38, the subject matter of any one or more of Examples 36-37 optionally include wherein the processing system is configured to execute instructions to adjust the stimulation parameter set in response to anticipated medication wash-in and wash-out periods.

In Example 39, the subject matter of any one or more of Examples 36-38 optionally include wherein the processing system is configured to execute instructions to increase a parameter of the stimulation parameter set in response to a reduced symptom relief.

In Example 40, the subject matter of any one or more of Examples 36-39 optionally include wherein the processing system is configured to execute instructions to decrease a parameter of the stimulation parameter set in response to an emergence of at least one side effect.

In Example 41, the subject matter of any one or more of Examples 36-40 optionally include wherein the processing system is configured to execute instructions to receive a sleep indication or a wakefulness indication from the patient.

In Example 42, the subject matter of Example 41 optionally includes wherein the processing system is configured to execute instructions to adjust a parameter of the stimulation parameter set in response to receiving the sleep indication or the wakefulness indication from the patient.

In Example 43, the subject matter of any one or more of Examples 36-42 optionally include wherein the processing system is configured to execute instructions to decrease a parameter of the stimulation parameter set in response to an emergence of at least one side effect.

In Example 44, the subject matter of any one or more of Examples 36-43 optionally include wherein the processing system is configured to execute instructions to override the trigger signal after the specified period of time has occurred to return to using the stimulation parameter set.

In Example 45, the subject matter of any one or more of Examples 36-44 optionally include wherein the processing system is configured to execute instructions to prevent reactivation of a trigger signal within a specified period of time after receiving the trigger signal.

In Example 46, the subject matter of any one or more of Examples 36-45 optionally include wherein the processing system is configured to execute instructions to identify a repetition of trigger signals over a specified period of time and generate a schedule for the stimulation parameter set used to provide the stimulation therapy based on the identified repetition of trigger signals.

In Example 47, the subject matter of any one or more of Examples 36-46 optionally include wherein the processing system is configured to execute instructions to display an adjustment to the stimulation parameter set to the patient for approval prior to implementing the adjustment.

In Example 48, the subject matter of any one or more of Examples 36-47 optionally include wherein the processing system is configured to execute instructions to decrease a parameter of the stimulation parameter set in response to an emergence of at least one side effect.

In Example 49, the subject matter of any one or more of Examples 36-48 optionally include wherein the processing system is configured to execute instructions to disable additional trigger signals during a specified period of time.

In Example 50, the subject matter of any one or more of Examples 36-49 optionally include wherein the processing system is configured to execute instructions to generate a schedule for the stimulation parameter set based on a history of consistent receipt of trigger signals during a specified period of time or in a specified pattern.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7 illustrates, by way of example and not limitation, a method for patient guided neuromodulation therapy.

FIG. 8A illustrates, by way of example and not limitation, a method for entering patient data for patient guided neuromodulation therapy.

FIG. 8B illustrates, by way of example and not limitation, a diagram showing timepoints for medication dosages and queries for patient guided neuromodulation therapy.

FIG. 8C illustrates, by way of example and not limitation, a diagram showing timepoints for non-medicated patients for patient guided neuromodulation therapy.

FIG. 9 illustrates, by way of example and not limitation, a flowchart showing phases of monitoring and providing triggers for patient guided neuromodulation therapy.

FIG. 10 illustrates, by way of example and not limitation, a flowchart of a method for patient guided neuromodulation therapy.

FIG. 11 illustrates, by way of example and not limitation, a flowchart of a method for patient triggered stimulation adjustment for patient guided neuromodulation therapy.

FIGS. 12A-12C each illustrate, by way of example and not limitation, graphs associated with medication and stimulation amplitude for patient guided neuromodulation therapy.

FIG. 13 illustrates, by way of example and not limitation, a flowchart of a method for trigger activated stimulation adjustment for patient guided neuromodulation therapy.

FIG. 14 illustrates, by way of example and not limitation, a neuromodulation therapy system, which may be used to deliver DBS.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Movement disorders are often treated with a combination of medication and stimulation. For example, patients can be implanted with a deep brain stimulation (DBS) implant and also administered medication to alleviate symptoms. During a 24 hour cycle of taking medications, depending on their pharmacodynamics and/or pharmacokinetcs, medication concentrations can wax and wane over time and their therapeutic efficacy can change accordingly, resulting in a high state of medication or a low state of medication. Physicians can balance treatments of electrostimulation and/or medication so that when the patient is in a low state of medication, the system adequately addresses the patient's symptoms by supplementing with electrostimulation. Likewise, when the patient is in a high state of medication the electrostimulation provided to the patient can be adjusted (e.g., lowered) so that the patient is not experiencing excessive electrostimulation and/or unwanted side effects. Some patients may experience fluctuations in disease management that are significant enough for the patient to return to the clinic and obtain additional therapy management from their clinicians to remedy. However, the programming of such electrostimulation and/or medication is performed in-clinic and may not be adjustable once a patient leaves the clinic.

In order to address these and other issues, a patient guided neuromodulation therapy can be used to obtain patient feedback and adjust the neuromodulation therapy accordingly. For example, a patient can be queried at specified time intervals or during specified events (e.g., when a patient takes a dose of medication) in order to determine how effective the neuromodulation therapy has been during a specified period of time. As efficacy of the neuromodulation therapy can be affected by a number of factors, the stimulation and/or medication can be adjusted based on the patient feedback in real time.

A patient device, such as a mobile or cellular phone, may include an application used to query the patient for the therapy information. The application may request the patient, a caregiver, a physician, or any connected system to create a medication schedule and generate appropriate alarms to remind the patient to take their medication. The application may request an average wake up time of the patient and/or a sleep time for the patient. In some examples, when the application provides an alarm for the patient to take their medication, the application may ask the patient a question or series of questions. These questions can include asking what percentage of a period of time (e.g., a period of time such as between a last dose and the next dose) did the patient feel that their symptoms were well managed without negative side effects or motor fluctuations. Further, the questions can include a sliding scale multiple choice approach to determine whether symptoms and/or side effects have increased or decreased during a period of time. These one or more questions may be repeated one hour before the patient goes to bed, at a time that a dose is administered, etc. In order to reduce question fatigue (e.g., where a patient is less responsive to questions due to the questions being asked too frequently), the questions may occur during a brief period of time, such as one week before seeing a physician and/or one week after seeing the physician.

The adjustments to the neuromodulation therapy may be in response to a triggered event indicated by a patient. The adjustment may last for a specified period of time, such as 24 hours. The triggered event may be a patient indicating that the patient took a dose of medication. The triggered event may be used to determine a wash-in period for the medication and a point in time that the stimulation should be reduced for a period of time (such as 1-4 hours) in order to avoid overlapping the high medication state and a higher stimulation state, which may lead to side effects. After the period of time, the stimulation therapy may return back to a baseline level. In this way, the neuromodulation therapy may be adjusted outside of the clinical setting based on patient guided feedback and be responsive to the experience of the patient rather than a set, non-adjusted management plan that is static and sometimes ineffective or less effective than anticipated.

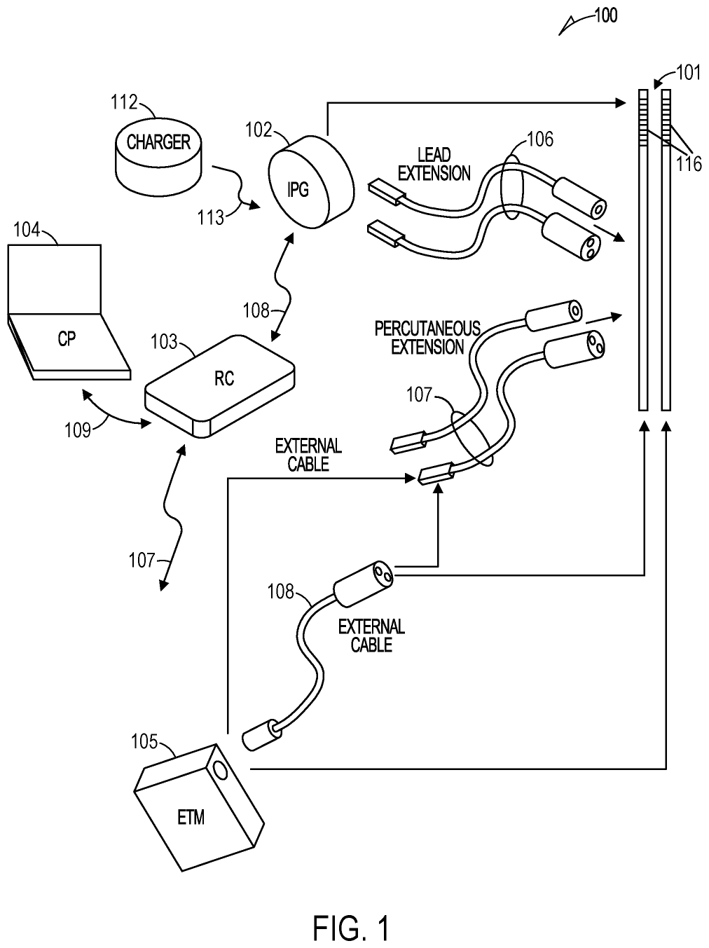

FIG. 1 illustrates, by way of example and not limitation, an electrical stimulation system 100, which may be used to deliver DBS. The electrical stimulation system 100 may generally include one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads may be provided, including only one, as long as the number of electrodes is greater than two (including the IPG case function as a case electrode) to allow for lateral steering of the current. Alternatively, a surgical paddle lead may be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 may likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters may be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies may be performed by executing software instructions contained within the CP 104. Alternatively, such programming methodologies may be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

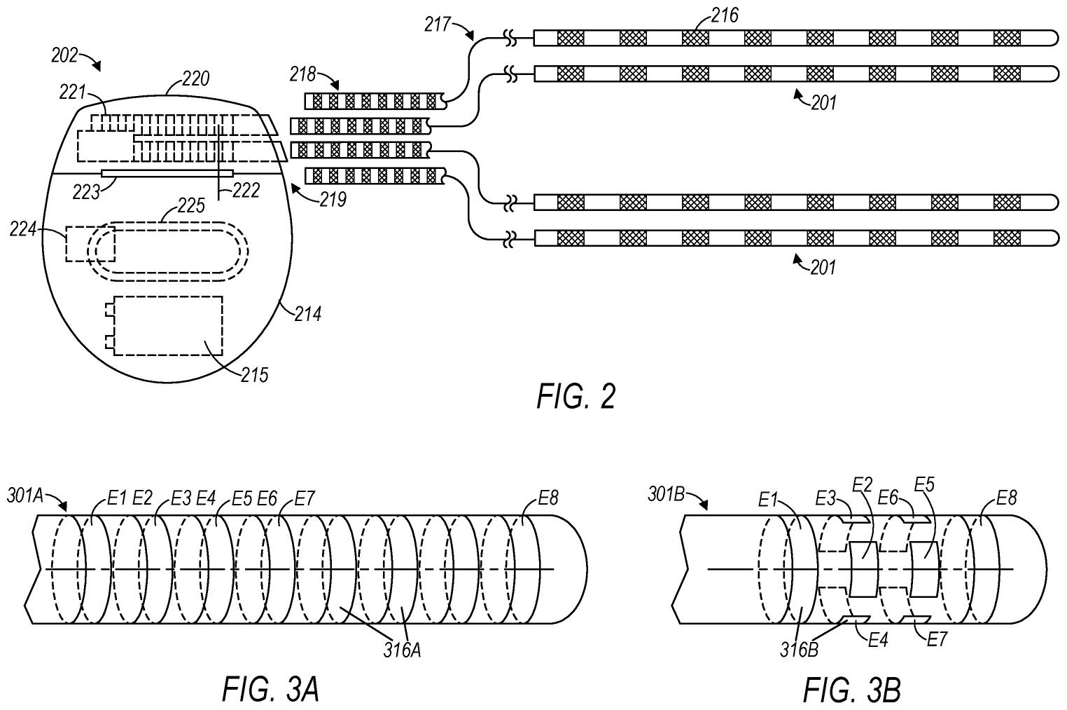

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more rows of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore may vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue; a comparison of a set threshold value; user-determined values or thresholds, or a change/delta from a prior measurement. Each of the electrodes may either be used (an active electrode) or unused (OFF). When the electrode is used, the electrode may be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a non-directional lead 301A with electrodes 316A disposed at least partially about a circumference of the non-directional lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a directional lead 301B with electrodes 316B including ring electrodes such as E1 at a proximal end and E8 at the distal end. Additionally, the lead 301 also includes a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide more-superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

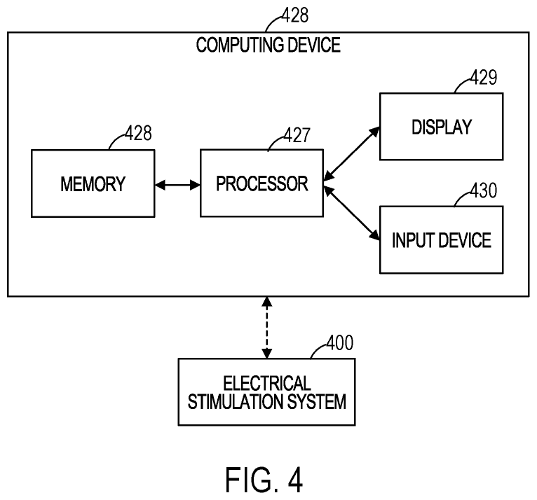

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease.

In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, a closed-loop algorithm for determining or updating stimulation parameters may be implemented in a mobile device, such as a smartphone, which is connected to the IPG or an evaluating device (e.g., a wristband or watch). These devices may also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and may include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

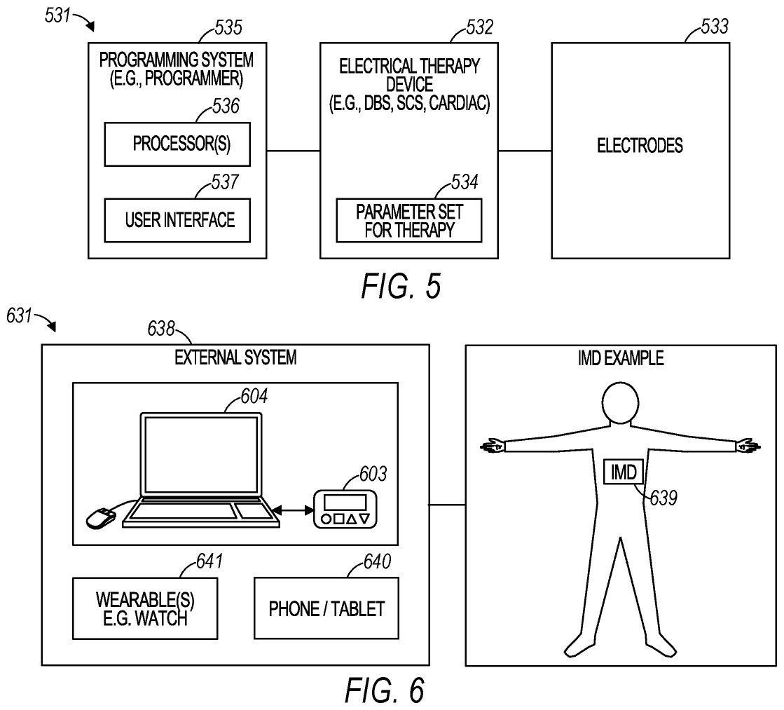

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameter sets through a computerized programming system to allow the optimum modulation parameters to be determined based on patient feedback or other means and to subsequently program the desired modulation parameter sets.

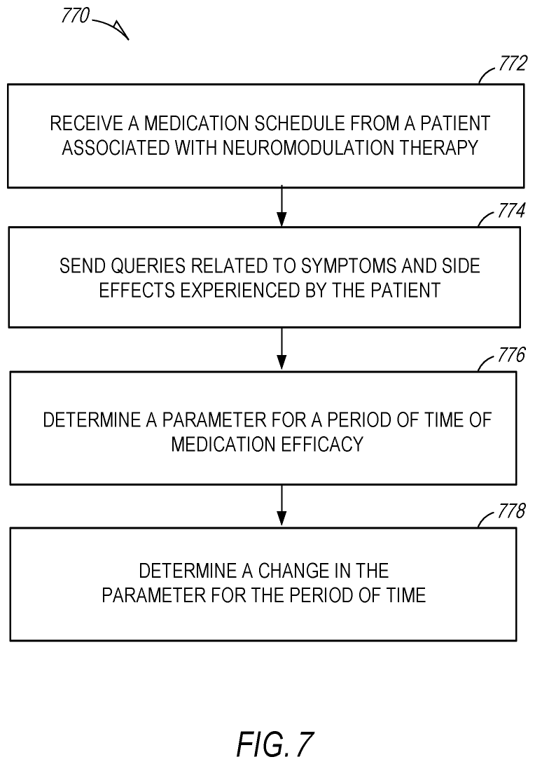

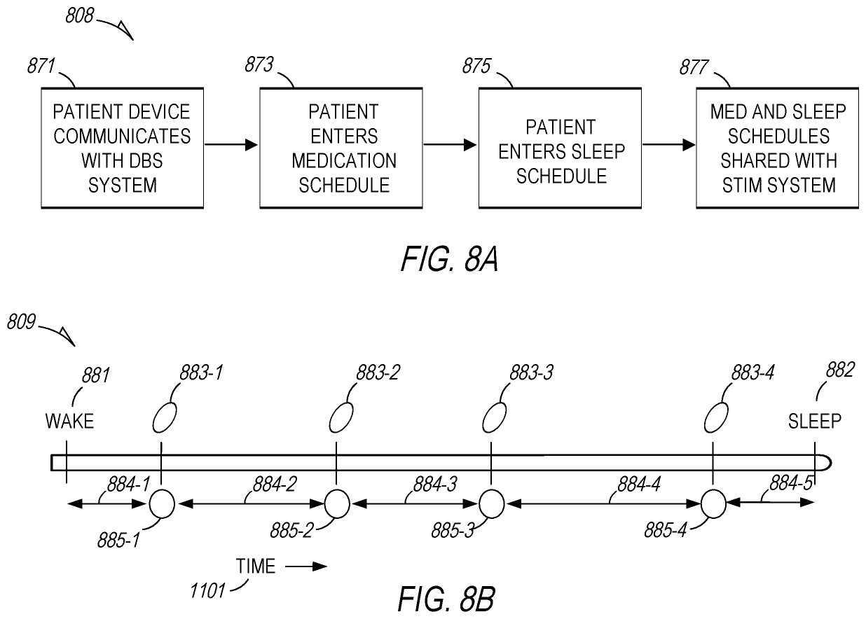

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

FIG. 7 illustrates, by way of example and not limitation, a method 770 for patient guided neuromodulation therapy. At 772, the method 770 may include receiving a medication schedule from a patient associated with neuromodulation therapy. The neuromodulation therapy may be performed using a processing system to deliver the stimulation therapy to a patient. A memory may be coupled to the processing system and may store instruction that, when executed by the processing system, cause the processing system to perform a number of functions. Instructions executable by the processing system may cause the processing system to program at least one medical device with a selected stimulation parameter to deliver the electrostimulation. The number of functions may include the performance of aspects of the method 770.

Although a medication schedule may assume that a dose response relationship is predictable or static (e.g., on-time for the medication reaches a maximum level and steadily falls “OFF” before the patient takes another dose), fluctuations may occur, especially at advanced stages of a disease. These fluctuations may occur between consecutive doses of medications and be based on a number of factors that make the dose to response relationship less predictable. These number of factors may include a delayed “ON,” a rapid wearing “OFF” towards the end of a dose time period, an unpredictable “ON/OFF” occurrence, a partial “ON,” and/or a complete dose failure (e.g., no “ON” at all). These factors may occur for any number of reasons such as sudden or gradual events affecting the patient's mood, various states of consciousness, such as sleep or drowsiness, interactions with other medications that the patient uses, or may occur for reasons that are unknown. Either way, it may be helpful to determine the experience of the patient in order to determine when these events or unpredictable behaviors of the medication or therapy are occurring. In some examples, a sleep schedule may be received from the patient. The sleep schedule may be used to determine the parameter for the period of time.

At 774, the method 770 may include sending queries related to symptoms and side effects experienced by the patient. The method 770 may include sending at least one query associated with determining a percentage of time while the patient was awake during a period of time that the patient experienced a change in side effects or symptoms. The change in side effects or symptoms can include increase side effects or symptoms, reduced side effects or symptoms, or no side effects or symptoms. The queries related to the symptoms and the side effects may be sent at specified time intervals. In some examples, the queries related to the symptoms and the side effects may be sent during a one week period prior to or a one week period subsequent to the patient seeing a physician. The method 770 may include communicating with a caregiver of the patient to receive caregiver feedback regarding the medication efficacy of the patient. In some examples, for patients not on a schedule for querying based on another criteria, the patient may be asked what percentage of time the patient was awake during a week did the patient feel his or her symptoms were well managed without negative side effects. For patients receiving medication, and for caregivers of the patient, the query may be asked every other week, since the patient application use may be higher in order to respond to the alarms and caregivers may be motivated to provide information. For patients not taking medication, the query may be asked once a month due to their low use of the patient application.

At 776, the method 770 may include determining a parameter for a period of time of medication efficacy. In some examples, the parameter is an average medication efficacy over the period of time. In some examples, reports may be provided that include at least one of an average on-time per dose, a daily average on- time, a weekly average on-time, a monthly average on-time, and a variance of at least one of the previously mentioned averages. The term “on-time” may refer to a period of time that the medication is “ON” or providing at least some effectiveness. In some examples, the parameter is a percentage of time. The method 770 may include determining that the patient experienced at least one side effect or at least one symptom during the percentage of time during the period of time. The method 770 may include sending a query to determine the percentage of time at least one hour before the patient is to go to sleep.

At 778, the method 770 may include determining a change in the parameter for the period of time. The method 770 may include determining that the change in the parameter exceeds a threshold change and, in response to the parameter exceeding the threshold change, recommending an adjustment to the neuromodulation therapy. The method 770 may include providing a recommendation based on a response to the queries that indicates the patient is trending in a positive direction or a negative direction. The method 770 may include receiving responses to the queries and analyzing the responses to determine the change in the parameter. Patterns in the parameter of the medication efficacy may identified during different periods of time. The method 770 may include determining a threshold change for the parameter of the medication efficacy and sending additional queries to the patient when the parameter exceeds the threshold change in the parameter. A frequency at which the queries are sent to the patient may be based on a proximity of a scheduled physician visit or upon request by a user associated with the patient. For example, as a scheduled physician visit gets closer in time, the frequency of the queries may increase in order to provide additional data to the physician in closer time proximity to the appointment. In some examples, the queries may be sent every other week, once a month for patients who are not administered medication, or at any specified frequency, as frequencies are not limited to those described herein.

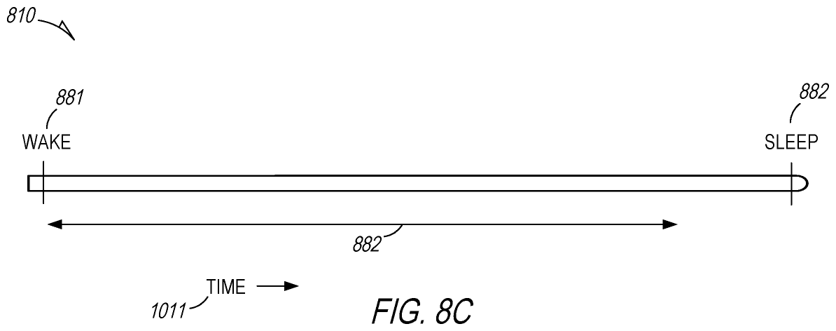

FIG. 8A illustrates, by way of example and not limitation, a method 808 for entering patient data for patient guided neuromodulation therapy. At 871, the method 808 may include a patient device communicating with a deep brain stimulation (DBS) system. The patient device may be a mobile or cellular phone, a computing device, etc. The patient device may include an application that is used to communicate with the DBS system. The DBS system may be a system such as is described above in association with FIGS. 1-6. For example, the patient device may be an external system, such as the external system 638 in FIG. 6. The DBS system may be similar to the electrical stimulation system 400 shown in FIG. 4.

At 873, the method 808 may include a patient entering a medication schedule into the patient device. The medication schedule may indicate time points throughout the day that the patient administers a dosage of medication. The time points may be averaged time points or may include a range of times that the patient administers the dosages.

At 875, the method 808 may include entering a sleep schedule for the patient into the patient device. The sleep schedule may indicate a time point when the patient goes to sleep and a time point when the patient wakes up. In some examples, additional data may be supplemented using wearables or other sensors measuring circadian patterns from the body using an EKG, heart rate, breathing rate, etc. The time points may be an averaged time point for going to sleep and waking up or a range of time points. The sleep schedule may indicate a period of time that the medication is not administered. The sleep schedule may indicate a low point of medication in the patient's body due to a night of sleep where the medication is not administered.

At 877, the method 808 may include sharing the medication schedule and the sleep schedule with a stimulation system (e.g., a DBS system such as ESS 400 in FIG. 4, ETD 532 in FIG. 5, or some other system). The schedules may be shared with a CP (e.g., CP 104 in FIG. 1), an RC (e.g., RC 103 in FIG. 1), a user application, etc. In this way, the medication schedule and the sleep schedule is communicated from the patient, or another representative of the patient, to the DBS system in order to provide the neuromodulation therapy to the patient or to assist in doing so.

FIG. 8B illustrates, by way of example and not limitation, a diagram 809 showing timepoints for medication dosages and queries for patient guided neuromodulation therapy. A wake time point 881 indicates when a patient woke from sleep. A number of dosages of medication 883-1 to 883-4 indicate a time point when medication was administered to the patient. A sleep time point 882 indicates when the patient went to sleep. A first time period 884-1 occurs between when the patient woke up 881 and a first dose 883-1, a second time period 884-2 occurs between the first dose 883-1 and a second dose 883-2, and so forth with a third time period 884-3, a fourth time period 884-4, and a fifth time period 884-5. A first query time point 885-1 indicates when a query is sent to the patient. The first query time point 885-1 is at a same time point as the first dose 883-1 was taken by the patient. In this way, the administering of the first dose 883-1 coincides with the first query being sent, at first query time point 885-1, since the reminder to take the first dose 883-1 will already have the patient using the application to receive the reminder. A second query time point 885-2 coincides with the second dose 883-2, and so forth, for a third 885-3 and fourth 885-4 query time point.

FIG. 8C illustrates, by way of example and not limitation, a diagram 810 showing timepoints for non-medicated patients for patient guided neuromodulation therapy. The diagram 810 illustrates a period of time 882 (with time 1011 being on the x-axis) where a patient is not receiving medication. The diagram 810 shows a wake time point 881 and a sleep time point 882, similar to FIG. 8B, but does not show a query time point. A period of time 882 from wakefulness 881 to sleep 882 may occur without sending a query to the patient. This is due to the lack of reminders for taking medication and/or a lower level of interaction with the application for the patient, thereby trying to avoid adding queries into the application during a period of time 882 when such queries may not be necessary or may not provide useful information.

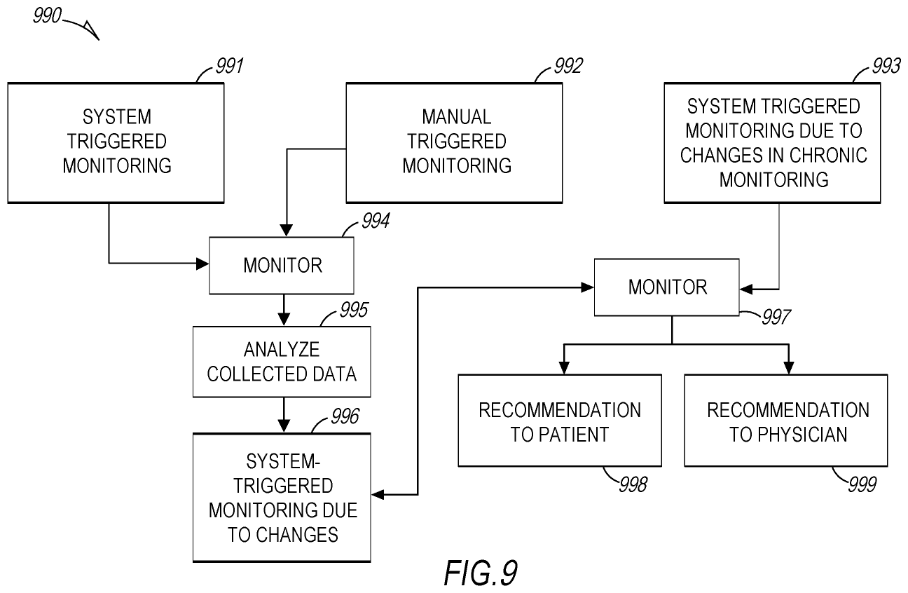

FIG. 9 illustrates, by way of example and not limitation, a flowchart 990 showing phases of monitoring and providing triggers for patient guided neuromodulation therapy. At 991, the flowchart 990 may include a system-triggered monitoring for an upcoming visit to a physician by the patient.

At 992, the flowchart 990 may include a manual-triggered monitoring or optimization by an approved user (either a patient or physician). The manual-triggered monitoring may include providing queries to the patient at an increased frequency or at specified times in order to gather and monitor information about the therapy. The user may set a custom time interval for the patient. Time intervals may include when an individual is queried throughout the day, what days per week the querying will occur, and how many weeks the query may continue (or minimum data points that may be used for providing data). For either of 991 or 992, the patient may be monitored, at 994, in such a way as is described in FIGS. 8A-8B, such as providing queries to the patient and maintaining data on side effects and symptoms.

At 995, the flowchart 990 may include analyzing collected data from the monitoring. The analysis of the collected data may include identifying significant changes or trends in the experience of the patient, such as changes or trends in symptoms or side effects. The analyzed data may be sent to the connected systems for providing DBS therapy. Analyzing the data may include determining average on-time for each dose, average on-time percentage per waking hour, daily average on-time, weekly average on-time, monthly average on-time, and/or a variance of each recording from the averages just mentioned. All of the above may be analyzed on the back end and may be available in reports to any connected systems. Once enough samples have been collected to establish an average, trends may be observed in consecutive time points. As part of the reports, an overall indication of change may be presented as well as an analysis of the trends. Recommendations may further be made that the patient appears stable or to be trending in a positive or negative direction based on the provided data. When a negative change is observed, the system may generate an alternative recommendation based on previously collected data from the same or other patients.

At 996, the flowchart 990 may include system-triggered monitoring due to the changes. At 993, the flowchart 990 may include system-triggered monitoring or optimization due to identified changes in more chronic monitoring. The following are threshold for a reaction by the system to change. A significant change threshold may refer to a change in on-time that is significantly outside the average and may be noted as a significant change (if negative) and may be reacted to quickly. A trending negative change threshold may refer to a change in on-time that is trending outside the average but may not reach a threshold of significant change and may request more data points to confirm the trend before a reaction is triggered. A user set change threshold may refer to a change in on-time that exceeds a user defined threshold for intervention. Triggered reactions may include informing connected users. For example, a query workflow may be triggered to increase a certainty or confidence in the change. Current queries may be refined to gather more information on symptoms and side effect management. For users with multiple time points of query (e.g., medicate users) where only one timepoint may be impacted, analysis may be limited or prioritized around an impacted timepoint only.

In response to the additional monitoring, the system may evaluate the available therapy programs being used and determine if a change in any of the existing programs has a potential to address the reduced on-time, increased side effects, reduction in symptom management, etc. Specifically, the system may evaluate a confidence level in the source of the indicated reduced on-time for the medication. If a program is identified that addressed the reduced on-time (e.g., increases the on-time), the recommendation may be sent to users to apply the change. In some examples, the system may be allowed to make certain recommendations to particular users (e.g., patients versus physicians). In these situations, the recommendation may be identified and sent only to the user with the appropriate permission. As illustrated, at 998, a recommendation is sent to a patient and, at 999, a recommendation may be sent to a physician. If a patient cannot be provided with a recommendation, due to limited permission, the triggered reactions may be delayed a period of time. For example, the triggered reactions may be delayed to occur no more than 1 month before a scheduled physician visit. In this example, all recommendations may be sent to the clinician programmer and be available when the IPG (e.g., IPG 102 in FIG. 1) is connected in clinic.

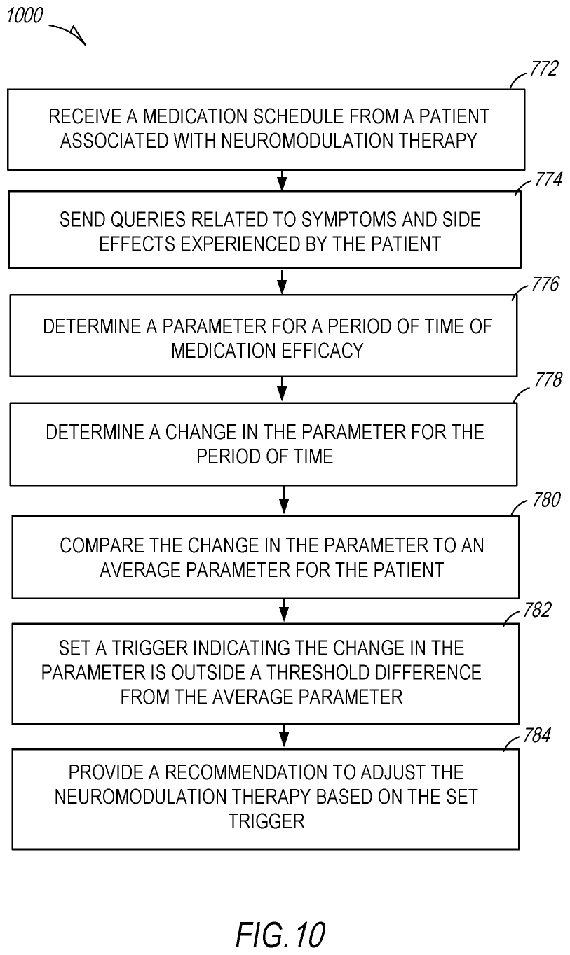

FIG. 10 illustrates, by way of example and not limitation, a flowchart 1000 of a method for patient guided neuromodulation therapy. The receive 772, send 774, determine 776, and determine 778 phases of the flowchart 1000 are similar to those described in association with FIG. 7. In addition, the flowchart 1000, at 780, may include comparing the change in the parameter to an average parameter for the patient. At 782, the flowchart 1000 may include setting a trigger indicating the change in the parameter is outside a threshold difference from the average parameter. At 784, the flowchart 1000 may include providing a recommendation to adjust the neuromodulation therapy based on the set trigger. As was described in association with FIG. 9, these thresholds and triggers help to determine whether to make a change or whether to maintain the neuromodulation therapy in a constant state until further verification is performed.

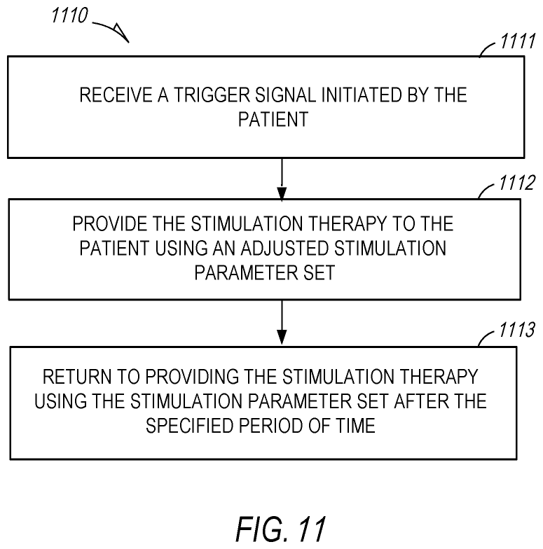

FIG. 11 illustrates, by way of example and not limitation, a flowchart 1110 of a method for patient triggered stimulation adjustment for patient guided neuromodulation therapy. At 1111, the flowchart 1110 may include receiving a trigger signal initiated by a patient. The trigger signal may be communicated through a button pressable by the patient when the patient has taken a dosage of medication. The system may be unaware of a medication schedule of the patient when the button is pressed by the patient. The trigger signal may initiate a change in the stimulation therapy due to an increasing effect of the medication on the patient as the medication washes in (e.g., a level of the medication in the patient's body increases).

At 1112, the flowchart 1110 may include providing the stimulation therapy to the patient using an adjusted stimulation parameter set. During the medication wash-in period, the stimulation parameters may be decreased in order to decrease the effects of the stimulation on the patient. A delay period could be initiated where an anticipated wash-in time occurs and then the stimulation parameters are gradually decreased (e.g., amplitude decreased) to a target value such that a maximal decrease coincides with a maximal wash-in of the medication (e.g., anticipated approximately 30 minutes after the trigger signal is sent, although times may vary). The adjusted stimulation parameter set may maintain at this lower threshold for a set period of time coinciding with the anticipated maximal state of medication. In some examples, the anticipated time would last for a span of approximately 1 to 4 hours.

In some examples, adjusting the stimulation parameter set may include decreasing at least one parameter of the stimulation parameter set. In some examples, adjusting the stimulation parameter set may include increasing at least one parameter of the stimulation parameter set. In some examples, adjusting the stimulation parameter set may include decreasing at least one parameter and increasing at least one parameter of the stimulation parameter set.

At 1113, the flowchart 1110 may include returning to providing the stimulation therapy using the stimulation parameter set after a specified period of time. At a time point when the span of time (approximately 1-4 hours) has passed, the stimulation parameter set used would increase back or return to a base level stimulation parameter set. Different medications may have different wash-in and wash-out temporal dynamics and different medications may require patient-specific amplitude adjustments. Further, for example, patient-specific adjustments to a stimulation parameter set may include increasing at least one parameter, decreasing at least one parameter, or increasing at least one parameter while decreasing at least one parameter.

While medication is a know driver of therapy changes for both movement disorders and pain, acute changes in symptom relief may occur as a result of external factors. During these times, a higher level of stimulation may help manage acute losses in symptom relief. However, it may be undesirable to maintain a stimulation at a higher level for an extended period of time for a variety of reasons. When a user indicates a reduced symptom relief, amplitude may instantly increase for some example programs. Therefore, therapy may be maintained at this level for 1-4 hours and then revert back to a base setting.

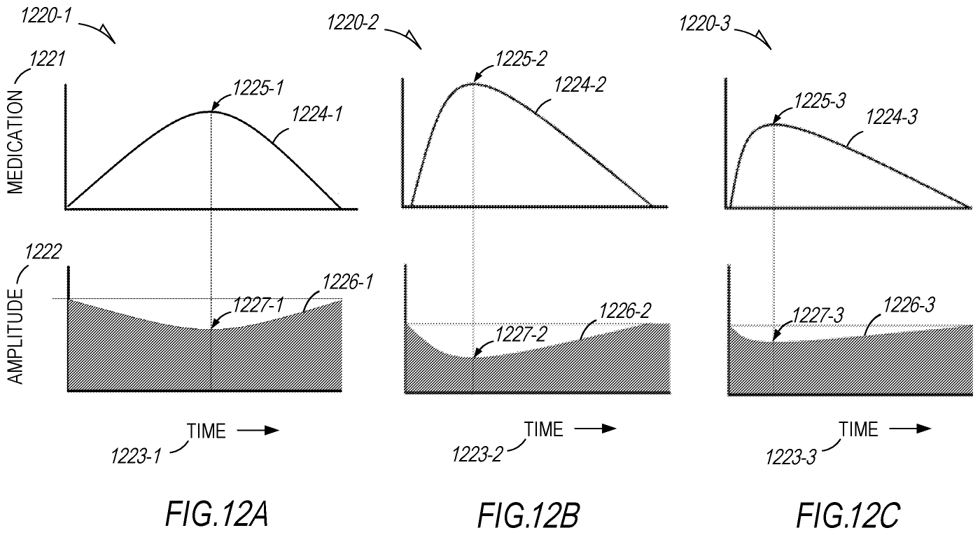

FIGS. 12A-12C each illustrate, by way of example and not limitation, graphs, 1220-1, 1220-2, 1220-3, associated with medication magnitude 1221 and amplitude 1222 for patient guided neuromodulation therapy. Different medications may have different temporal dynamics and thus may use different changes in stimulation amplitude over time. Temporal characteristics may be a function of a maximum effectiveness of the medication, differences in side effects evoked by the medication, as well as temporal dynamics of wash-in and wash-out of the medication. Stimulation amplitude may be compensated for depending on the medication dynamics. An appropriate amplitude may be superimposed and compared to a medication level and adjusted accordingly, as is illustrated in each of FIGS. 12A-12C.

FIG. 12A illustrates a level of medication 1224-1 over a period of time 1223-1 with a peak of medication 1225-1. A level of amplitude 1226-1 over the period of time 1223-1 is shown with a trough 1227-1 of amplitude. The peak of medication 1225-1 coincides in time with the trough of amplitude 1227-1, illustrating the opposing changes of the medication level and magnitude in order to provide the proper level of therapy without providing too much overlapping therapy of the stimulation or medication.

FIG. 12B, likewise, illustrates a level of medication 1224-2 over a period of time 1223-2 with a peak of medication 1225-2. A level of amplitude 1226-2 over the period of time 1223-2 is shown with a trough 1227-2 of amplitude. The peak of medication 1225-2 coincides in time with the trough of amplitude 1227-2, illustrating the opposing changes of the medication level and amplitude magnitude. In FIG. 12B, the peak and corresponding trough occurs earlier in time and to a greater degree, most likely due to a quicker and higher leveled wash-in of the medication with a quicker and lower decrease in the amplitude magnitude to compensate.

FIG. 12C, likewise, illustrates a level of medication 1224-3 over a period of time 1223-3 with a peak of medication 1225-3. A level of amplitude 1226-3 over the period of time 1223-3 is shown with a trough 1227-3 of amplitude. The peak of medication 1225-3 coincides in time with the trough of amplitude 1227-3, illustrating the opposing changes of the medication level and amplitude magnitude. In FIG. 12B, the peak and corresponding trough occurs earlier in time but with a lower peak of medication and smaller decrease in amplitude, most likely due to a quicker lower leveled wash-in of the medication with a quicker but more shallow decrease in the amplitude magnitude to compensate.

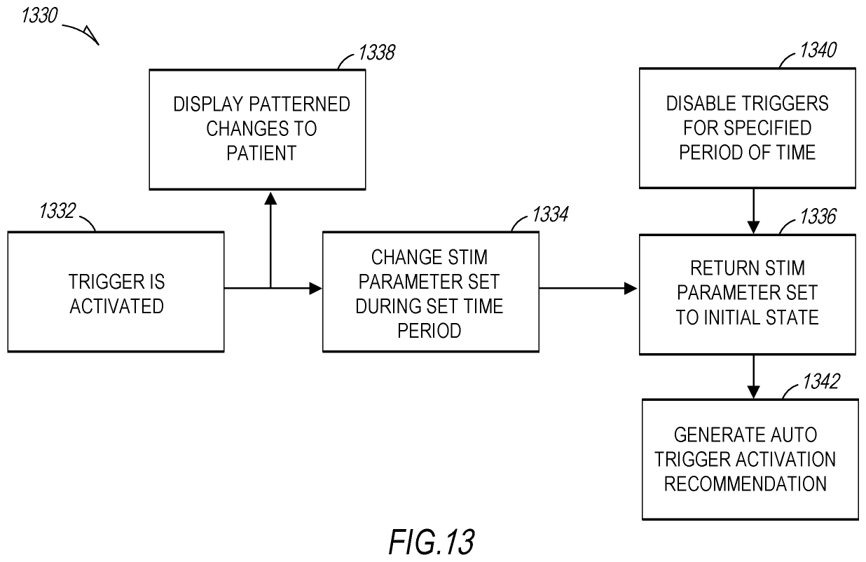

FIG. 13 illustrates, by way of example and not limitation, a flowchart 1330 of a method for trigger activated stimulation adjustment for patient guided neuromodulation therapy. At 1332, the flowchart 1330 may include activating a trigger. At 1338, the flowchart 1330 may optionally include displaying patterned changes to the patient. At 1334, the flowchart 1330 may include changing a stimulation parameter set during a set time period.

At 1336, the flowchart 1330 may include returning the stimulation parameter set to an initial state. For example, a trigger which overrides any other trigger would return the stimulator back to a stimulation state that had been predominantly used over a previous period of time (e.g., over the last week, the last 2 weeks, the last month, etc.). When stimulation has not been enable for a period of time (e.g,. a week), the stimulation may be changed to a level that was used most frequently. The above descriptions may rule out instances where stimulation was generated or applied due to triggering events.

At 1340, the flowchart 1330 may include disabling triggers for a specified period of time. In some examples, stimulation implemented in response to a trigger, such as increasing amplitude in response to high pain levels, may change stimulation in a way that is best implemented over a brief time period only. In these instances, when a trigger is used, it may lock out from repeated use in a set period of time consisting of hours or days. This may avoid overstimulation that may be counterproductive or make the side effects or symptoms worse. The patient may still be able to manually change stimulation to a same level applied by the program.

At 1342, the flowchart 1330 may include generating an auto-trigger activation recommendation. The recommendation may be based on a history of consistent triggering. As an example, within a period of time (e.g., a week or two), repeated triggers at a same time interval for consistent features such as medication or sleep and wake times may identify timed occurrences and create a schedule. When the schedule is created, instead of returning to a base feature, the schedule may shift between a minimum and maximum that would be utilized by the respective programs. For instance, the schedule may shift to a middle point rather than returning to a base.

In some examples, a trigger may be activated due to a response to wakefulness. Stimulation has been shown to help patients sleep, but may not be needed to maintain therapy at similar levels for when a patient is awake. For movement disorders, sensed controlled signals may be reduced during sleep, and some physicians may prefer reduced amplitudes during this time. However, when a patient wakes up, the patient may be in a medication low state. The stimulation of the patient may be increased to a higher amplitude to maintain therapy.

When a patient indicates that the patient is going to sleep, a program may wait a period of time (e.g., 30 minutes), and decrease the amplitude of stimulation. The amplitude may then be ramped back to a base level after between 4-8 hours of sleep. Similarly, for paint patients that sleep on their back, stimulation amplitude may be reduced for spinal cord stimulation (SCS) when sleep is indicated since the stimulator may be closer to the spinal cord at these times. The program may gradually return stimulation amplitude back to the base setting after between 4-8 hours. A second trigger may be used for when a patient indicates that they are awake. In this instance, the stimulation may then be ramped back up over a short period of time (e.g., from 1 to 10 minutes) to the base stimulation level).

In some examples, a patient may indicate, through a trigger or button to press, that the patient is experiencing a side effect. When the patient indicates this, the stimulation may be ramped down for a set period of time. The reduction of the stimulation may reduce the side effect experienced by the patient. In some examples, a patient may want to return to a baseline or previous therapy associated with a baseline or prior stimulation parameter set. The patient may then hit or press a button or other mechanism to indicate a desire to return to the baseline or prior stimulation parameter set. This may be triggered whenever the baseline therapy is not being applied. In this instance, regardless of other algorithms, indications, or therapies, the patient may be able to return to a known or previous therapy.

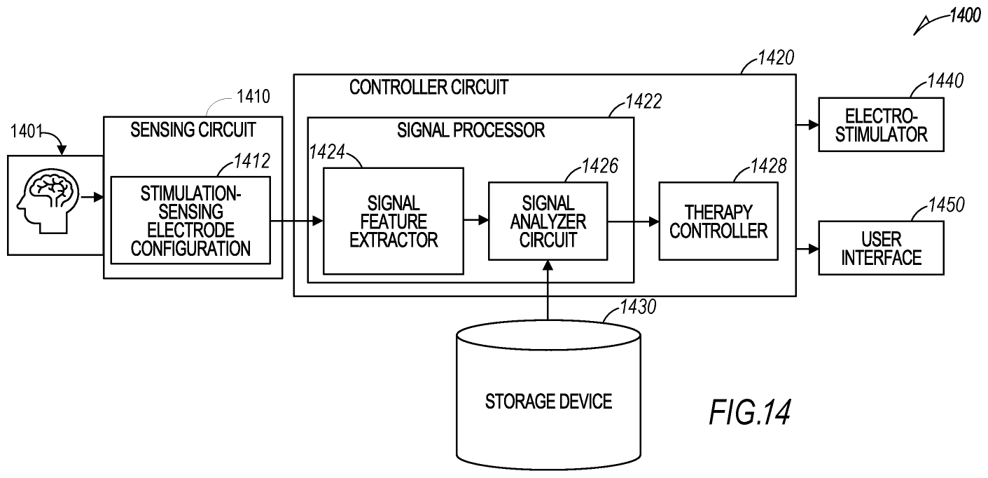

FIG. 14 illustrates, by way of example and not limitation, a neuromodulation therapy system, which may be used to deliver DBS based on evoked responses. The system 1400 includes a sensing circuit 1410, a controller circuit 1420, a storage device 1430, an electrostimulator 1440, and a user interface 1450. Portions of the system 1400 may be implemented in the IPG 102 or the CP 104.

The sensing circuit 1410 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 1440. The ring electrodes and/or the segmented electrodes may be configured as sensing electrodes for sensing ERs, or as stimulating electrodes for delivering electrostimulation pulses. The sensing circuit 1414 may sense ERs from one or more sensing electrodes on a lead placed at target issue (e.g., STN) of a patient 1401 in response to electrostimulation pulses delivered from a stimulating electrode at a stimulation site (e.g., a brain target). The ERs may be sensed in accordance with a stimulating-sensing electrode configuration 1412.

The controller circuit 1420 may include circuit sets comprising one or more other circuits or sub-circuits, such as a signal processor 1422 and a therapy controller 1428. The signal processor 1422 may further include a signal feature extractor 1424 and a signal analyzer circuit 1426. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation.

In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 1420 may be implemented as a part of a microprocessor circuit. The microprocessor circuit may be a dedicated processor such as a digital signal processor, application specific integrated circuit (ASIC), microprocessor, or other type of processor for processing information including physical activity information. Alternatively, the microprocessor circuit may be a general purpose processor that may receive and execute a set of instructions of performing the methods or techniques described herein.

The signal feature extractor 1424 may extract a signal feature from the filtered ER signal (e.g., the ER signal with the stimulation artifact removed). Examples of the signal features may include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, may be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1-P2 P2P” amplitude). The signal curve length may be measured as accumulated signal value differences of the sensed evoked response (or an epoch-averaged evoked response) over consecutive unit times (e.g., consecutive data sampling intervals) within the time window. The signal power may be measured as an area under the curve (AUC) of the sensed evoked response (or the epoch-averaged evoked response) within the time window. In some examples, the signal analyzer circuit 1426 may generate a spatial distribution of extracted signal features across sensing locations of the sensing electrodes.

The signal analyzer circuit 1426 may compare the filtered ERs, or signal features or a spatial distribution of signal features derived therefrom, to one or more states or conditions of the patient. In some examples, the states or conditions are used to adjust the neuromodulation parameters. In some examples, the signal analyzer circuit 1426 may accumulate the sensed ERs, and therefore determined states or conditions, obtained in multiple stimulation-ER recording sessions during which stimulation pulses are delivered via a particular stimulating electrode with varying stimulation parameter settings (e.g., stimulation amplitude, frequency, or pulse width), and compare the accumulated ERs (or signal features or a spatial distribution of signal features derived from the filtered ERs) to previously stored states or conditions. In an example, the states or conditions of the patient may be user-provided or loaded into the system for easy recognition. In another example, the state or condition may be associated with a target ER or target ER feature template representing a patient-specific ER feature or a population-based ER feature to electrostimulation of the neural target. In an example, the target ER template may be used to adjust the neuromodulation therapy to relieve symptoms or other goals such as co-therapy (e.g., leads that inject drugs or light), and side-effect avoidance. The signal analyzer circuit 1426 may determine a distribution of sensed ER features, compare the determined distribution of sensed ERs to the corresponding states or conditions to determine whether to adjust the neuromodulation therapy.

The therapy controller 1428 may generate a control signal to the electrostimulator 1440 to adjust the thresholds of the neuromodulation therapy based on the determined state or condition. The electrostimulator 1440 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 1428 may be implemented as a proportional integral (PI) controller, a proportional-integral-derivative (PID) controller, or other suitable controller that takes the comparison of the sensed ERs (or features or a distribution of the features thereof) to the corresponding states or conditions as a feedback on the adjustment of stimulation settings.

The electrostimulator 1440 may be an implantable module, such as incorporated within the IPG 102 in FIG. 1. Alternatively, the electrostimulator 1440 may be an external stimulation device, such as incorporated with the ETS 40. In some examples, the user may choose to either send a notification (e.g., to a smartphone with the patient) for a therapy reminder, or to automatically initiate or adjust neuromodulation therapy in accordance with the adjusted therapy setting. If an automatic therapy initiation is selected, the electrostimulator 1440 may deliver stimulation in accordance with the adjusted therapy setting.

In some examples, the therapy controller 1428 may generate a recommendation to the user to adjust the device setting (e.g., a programmable parameter of the electrostimulator 1440) to cause the sensed ERs to align with or to compare more favorably to one or more states or conditions of the patient. In some embodiments, the display may provide a suggestion to the user to adjust stimulation parameters to cause the since developed responses to more favorably compare to the state or condition or a transition to a different state or condition. In some examples, the therapy controller 1428 may determine or modify therapeutic stimulation settings based on the sense ERs or features thereof and the corresponding determined state or condition of the patient. The electrostimulator 1440 may deliver therapeutic stimulation (e.g., DBS) in accordance with the determined or modified therapeutic stimulation settings.

In some examples, the user interface 1450 allows a physician to remotely review therapy settings and treatment history, consult with the patient to obtain information including pain relief and SCS-related side effects or symptoms, perform remote programming of the electrostimulator 1440, or provide other treatment options to the patient. The user interface 1450 may allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program may include a set of stimulation parameters with respective pre-determined values. In some examples, the user interface 1450 may include a display to display textually or graphically information provided by the user via an input unit, and device settings including, for example, feature selection, sensing configurations, signal pre-processing settings, therapy settings, optionally with any intermediate calculations. In an example, the user interface 1450 may present to the user an “optimal” or improved therapy setting, such as determined based on a closed-loop or adaptive feedback control of electrostimulation based on a selected evoked response signal feature, in accordance with various embodiments discussed in this document. In some examples, the user may use the user interface 1450 to provide feedback on a neuromodulation therapy, including, for example, side effects or symptoms arise or persist associated with the neurostimulation, or severity of the symptom or a side effect.

As used herein, the term “machine readable medium” may include any medium that is capable of storing, encoding, or carrying instructions for execution by a processing device or machine and that causes the processing device or machine to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method, comprising:
receiving a medication schedule from a patient associated with neuromodulation therapy;
sending queries related to symptoms and side effects experienced by the patient;
determining a parameter for a period of time of medication efficacy; and
determining a change in the parameter for the period of time.

2. The method of claim 1, further comprising:
determining that the change in the parameter exceeds a threshold change; and
in response to the parameter exceeding the threshold change, recommending an adjustment to the neuromodulation therapy.

3. The method of claim 1, further comprising sending at least one query associated with determining a percentage of time while the patient was awake during a period of time that the patient experienced a change in side effects or symptoms.

4. The method of claim 1, further comprising sending the queries related to the symptoms and the side effects at specified time intervals.

5. The method of claim 1, further comprising:
receiving responses to the queries; and
analyzing the responses to determine the change in the parameter.

6. The method of claim 1, further comprising identifying patterns in the parameter of the medication efficacy during different periods of time.

7. The method of claim 1, wherein the parameter is an average medication efficacy over the period of time.

8. The method of claim 1, further comprising receiving a sleep schedule from the patient.

9. The method of claim 1, further comprising sending the queries at least 1 hour before the patient goes to bed.

10. The method of claim 1, further comprising sending the queries every other week.

11. The method of claim 1, further comprising sending the queries once a month for patients who are not administered medication.

12. The method of claim 1, further comprising receiving a response from the patient in response to the queries.

13. The method of claim 12, wherein the received response is used to determine one of: an average on-time for each dose administered to the patient, an average on-time percentage per waking hour, or a daily average on-time.

14. The method of claim 12, wherein the received response is used to determine one of: a weekly average on-time, a monthly average on-time, or a variance of each recording from a determined average,

15. The method of claim 1, further comprising providing a recommendation based on a response to the queries that indicates the patient is trending in a positive direction or a negative direction.

16. A non-transitory computer-readable medium storing instructions executable by a processor to:
receive a medication schedule from a patient associated with neuromodulation therapy;
send queries related to symptoms and side effects experienced by the patient;
determine a parameter for a period of time of medication efficacy; and
determine a change in the parameter for the period of time; and
compare the change in the parameter to an average parameter for the patient.

17. A system, comprising:
a processing system configured to deliver stimulation therapy to a patient; and
a memory coupled to the processing system, the memory storing instructions that, when executed by the processing system, cause the processing system to:
receive a medication schedule from a patient associated with neuromodulation therapy;
send queries related to symptoms and side effects experienced by the patient;
determine a parameter for a period of time of medication efficacy; and
determine a change in the parameter for the period of time.

18. The system of claim 17, wherein the processing system is configured for use to identify patterns in the parameter of the medication efficacy during different periods of time.

19. The system of claim 17, wherein the parameter is an average medication efficacy over the period of time.

20. The system of claim 17 wherein the processing system is configured for use to communicate with a caregiver of the patient to receive caregiver feedback regarding the medication efficacy of the patient.
